(19) United States
(12) Patent Application Publication
(10) Pub. No.: US 2006/0270655 A1
(43) Pub. Date: Nov. 30, 2006

(54) Combination therapy for treating obesity or maintaining weight loss

(76) Inventors: Andrew G. Swick, East Lyme, CT (US); Terrell A. Patterson, Salem, CT (US)

Correspondence Address: PFIZER INC., PATENT DEPARTMENT, MS8260-1611, EASTERN POINT ROAD, GROTON, CT, 06340, US

(21) Appl. No.: 11/435,695

(22) Filed: May 16, 2006

Related U.S. Application Data
(60) Provisional application No. 60/685,752, filed on May 27, 2005.
(60) Provisional application No. 60/697,516, filed on Jul. 7, 2005.

(51) Int. Cl. A61K 31/519; A61K 31/454; A61K 31/4172; A61K 31/4741; A61K 31/353

(52) U.S. Cl. 514/210.21; 514/326; 514/400; 514/262.1; 514/320; 514/302; 514/454

(57) ABSTRACT

Combination therapies for treating obesity or related eating disorders and/or reducing food consumption are described herein which comprises administering a therapeutically effective amount of a cannabinoid-1 (CB-1) receptor antagonist and an intestinal-acting microsomal triglyceride transfer protein inhibitor (MTPi) to an animal in need of such treatment. The CB-1 receptor antagonist and intestinal-acting MTPi may be administered separately or together.

This application claims the benefit of U.S. Provisional Application No. 60/685,752, filed on May 27, 2005, and 60/697,516, filed on Jul. 7, 2005, incorporated herein by reference in their entirety.

### FIELD OF THE INVENTION

The present invention relates to combination therapies for treating obesity or related eating disorders and/or reducing food consumption by administering a cannabinoid receptor-1 (CB-1) antagonist in combination with an intestinal-acting microsomal triglyceride transfer protein inhibitor (MTPi).

### BACKGROUND

Obesity is a major public health concern and is now recognized as a chronic disease that requires treatment to reduce its associated health risks. Although weight loss is an important treatment outcome, one of the main goals of obesity management is to improve cardiovascular and metabolic values to reduce obesity-related morbidity and mortality. It has been shown that 5-10% loss of body weight can substantially improve metabolic values, such as blood glucose, blood pressure, and lipid concentrations. Hence, it is believed that a 5-10% intentional reduction in body weight may reduce morbidity and mortality.

Currently available prescription drugs for managing obesity generally reduce weight by inducing satiety or decreasing dietary fat absorption. However, to date, the anti-obesity drugs available commercially provide only modest weight loss. The most successful drug regimens in humans have been combinations of phentermine and fenfluramine or of ephedrine, caffeine and/or aspirin. Each of these combinations have been discontinued due to safety concerns. Although investigations are on-going, there still exists a need for a more effective and safe therapeutic treatment for reducing or preventing weight-gain.

### SUMMARY OF THE INVENTION

The present invention provides a method for treating obesity or related eating disorders (preferably, reducing weight and/or maintaining weight loss (or preventing weight gain)) comprising the step of administering a therapeutically effective amount of a combination of a cannabinoid-1 (CB-1) receptor antagonist and an intestinal-acting microsomal triglyceride transfer protein inhibitor (MTPi) to an animal in need of such treatment. The CB-1 receptor antagonist and intestinal-acting MTPi may be administered separately or together. Preferably, the combination therapy is administered in conjunction with exercise and a sensible diet.

In another embodiment of the present invention, a method for reducing food consumption (including the desire to consume food) is provided comprising the step of administering a therapeutically effective amount of a combination of a cannabinoid-1 (CB-1) receptor antagonist and an intestinal-acting microsomal triglyceride transfer protein inhibitor (MTPi) to an animal in need of such treatment. The CB-1 receptor antagonist and intestinal-acting MTPi may be administered separately or together. Preferably, the combination therapy is administered in conjunction with exercise and a sensible diet.

The combination therapies described above may be administered as (a) a single pharmaceutical composition which comprises the CB-1 antagonist, the intestinal-acting MTPi and a pharmaceutically acceptable excipient, diluent, or carrier; or (b) two separate pharmaceutical compositions comprising (i) a first composition comprising the CB-1 antagonist and a pharmaceutically acceptable excipient, diluent, or carrier, and (ii) a second composition comprising the intestinal-acting MTPi and a pharmaceutically acceptable excipient, diluent, or carrier. The pharmaceutical compositions may be administered simultaneously or sequentially and in any order.

In another embodiment of the present invention, a pharmaceutical composition is provided comprising (i) a CB-1 receptor antagonist; (ii) a intestinal-acting MTPi; and (iii) a pharmaceutically acceptable excipient, diluent, or carrier, wherein the amount of CB-1 receptor antagonist is from about 1.0 mg to about 100 mg (preferably from about 1.0 mg to about 50 mg, more preferably from about 2.0 mg to about 40 mg, most preferably from about 5.0 mg to about 25 mg) and the amount of intestinal-acting MTPi is typically from about 0.05 mg to about 50 mg (preferably from about 0.5 mg to about 30 mg, more preferably from about 0.5 mg to about 20 mg, most preferably from about 1.0 mg to about 15 mg.

In yet another aspect of the present invention, a pharmaceutical kit is provided for use by a consumer to treat obesity and related eating disorders. The kit comprises a) a suitable dosage form comprising a CB-1 antagonist and an intestinal-acting MTPi; and b) instructions describing a method of using the dosage form to treat obesity and/or related eating disorders and/or reducing food consumption.

In yet another embodiment of the present invention is a pharmaceutical kit comprising: a) a first dosage form comprising (i) a CB-1 antagonist and (ii) a pharmaceutically acceptable carrier, excipient or diluent; b) a second dosage form comprising (i) an intestinal-acting MTPi and (ii) a pharmaceutically acceptable carrier, excipient or diluent; and c) a container.

### DEFINITIONS

As used herein, the phrase “therapeutically effective amount” means an amount of the combination of compounds of the present invention that (i) treats the particular disease (including conditions or disorders thereof), (ii) attenuates, ameliorates, or eliminates one or more symptoms of the particular disease, or (iii) prevents or delays the onset of one or more symptoms of the particular disease described herein (e.g., reduces food intake or the desire to consume food). The terms “treating”, “treat”, or “treatment” also embraces preventative (i.e., weight maintenance) treatment.

The term “animal” refers to humans (male or female), companion animals (e.g., dogs, cats and horses), food-source animals, zoo animals, marine animals, birds and other similar animal species. “Edible animals” refers to food-source animals such as cows, pigs, sheep and poultry. Preferably, the animal is human or a companion animal (preferably, the companion animal is a dog), more preferably, the animal is human (man and/or woman).

The phrase “pharmaceutically acceptable” indicates that the substance or composition must be compatible chemically and/or toxicologically, with the other ingredients comprising a formulation, and/or the mammal being treated therewith.

The term “antagonist” includes both full antagonists and partial antagonists, as well as inverse agonists.

The term “food” refers to food or drink for human or other animals' consumption.

### BRIEF DESCRIPTION OF THE FIGURES

FIG. 1 illustrates the decreased food intake observed for the combination of 10 mg/kg of Compound A and 3 mg/kg of Dirlotapide as compared to vehicle (no drug), 10 mg/kg of Compound A alone and 3 mg/kg of Dirlotapide alone.

FIG. 2 illustrates the decreased food intake observed for the combination of 10 mg/kg of Compound A and 10 mg/kg of Dirlotapide as compared to vehicle (no drug), 10 mg/kg of Compound A alone and 10 mg/kg of Dirlotapide alone.

FIG. 3 illustrates the decreased food intake observed for the combination of 30 mg/kg of Compound A and 3 mg/kg of Dirlotapide as compared to vehicle (no drug), 30 mg/kg of Compound A alone and 3 mg/kg of Dirlotapide alone.

FIG. 4 illustrates the decreased food intake observed for the combination of 30 mg/kg of Compound A and 10 mg/kg of Dirlotapide as compared to vehicle (no drug), 30 mg/kg of Compound A alone and 10 mg/kg of Dirlotapide alone.

### DETAILED DESCRIPTION

Applicants have discovered that significant reductions in food intake can be achieved by administering a CB-1 receptor antagonist in combination with an intestinal-acting MTP inhibitor. Preferably, the combination therapy is administered in conjunction with exercise and a sensible diet.

Cannabinoid-1 (CB-1) Receptor Antagonists:

As used herein, the term “CB-1 receptor” refers to a G-protein coupled type 1 cannabinoid receptor. Preferably, the CB-1 receptor antagonist is selective to the CB-1 receptor. “CB-1 receptor selective” means that the compound has little or no activity to antagonize the cannabinoid-2 receptor (CB-2). More preferably, the CB-1 antagonist is at least about 10 fold more selective for the CB-1 receptor in comparison to the CB-2 receptor. For example, the inhibitory concentration (IC₅₀) for antagonizing the CB-1 receptor is about 10 or more times lower than the IC₅₀ for antagonizing the CB-2 receptor. Bioassay systems for determining the CB-1 and CB-2 binding properties and pharmacological activity of cannabinoid receptor ligands are described by Roger G. Pertwee in “Pharmacology of Cannabinoid Receptor Ligands” Current Medicinal Chemistry, 6, 635-664 (1999) and in WO 92/02640 (U.S. application Ser. No. 07/564,075 filed Aug. 8, 1990, incorporated herein by reference).

Suitable CB-1 receptor antagonists include compounds disclosed in U.S. Pat. Nos. 5,462,960; 5,596,106; 5,624,941; 5,747,524; 6,017,919; 6,028,084; 6,432,984; 6,476,060; 6,479,479; 6,518,264; and 6,566,356;

U.S. Patent Publication Nos. 2003/0114495; 2004/0077650; 2004/0092520; 2004/0122074; 2004/0157838; 2004/0157839; 2004/0214837; 2004/0214838; 2004/0214855; 2004/0214856; 2004/0058820: 2004/0235926; 2004/0248881; 2004/0259887; 2005/0080087; 2005/0026983 and 2005/0101592;

PCT Patent Publication Nos. WO 03/075660; WO 02/076949; WO 01/029007; WO 04/048317; WO 04/058145; WO 04/029204; WO 04/012671; WO 03/087037; WO 03/086288; WO 03/082191; WO 03/082190; WO 03/063781; WO 04/012671; WO 04/013120; WO 05/020988; WO 05/039550; WO 05/044785; WO 05/044822; WO 05/049615; WO 05/061504; WO 05/061505; WO 05/061506; WO 05/061507; and WO 05/103052: and

U.S. Provisional Application Ser. Nos. 60/673,535 filed on Apr. 20, 2005; and 60/673,546 filed on Apr. 20, 2005.

All of the above patents and patent applications are incorporated herein by reference.

Preferred CB-1 receptor antagonists for use in the methods of the present invention include: rimonabant (SR141716A also known under the tradename Acomplia™) is available from Sanofi-Synthelabo or can be prepared as described in U.S. Pat. No. 5,624,941; N-(piperidin-1-yl)-1-(2,4-dichlorophenyl)-5-(4-iodophenyl)-4-methyl-1H-pyrazole-3-carboxamide (AM251) is available from Tocris™, Ellisville, Mo.; [5-(4-bromophenyl)-1-(2,4-dichloro-phenyl)-4-ethyl-N-(1-piperidinyl)-1H-pyrazole-3-carboxamide] (SR147778) which can be prepared as described in U.S. Pat. No. 6,645,985; N-(piperidin-1-yl)-4,5-diphenyl-1-methylimidazole-2-carboxamide, N-(piperidin-1-yl)-4-(2,4-dichlorophenyl)-5-(4-chlorophenyl)-1-methylimidazole-2-carboxamide, N-(piperidin-1-yl)-4,5-di-(4-methylphenyl)-1-methylimidazole-2-carboxamide, N-cyclohexyl-4,5-di-(4-methylphenyl)-1-methylimidazole-2-carboxamide, N-(cyclohexyl)-4-(2,4-dichlorophenyl)-5-(4-chlorophenyl)-1-methylimidazole-2-carboxamide, and N-(phenyl)-4-(2,4-dichlorophenyl)-5-(4-chlorophenyl)-1-methylimidazole-2-carboxamide which can be prepared as described in PCT Patent Publication No. WO 03/075660; the hydrochloride, mesylate and besylate salt of 1-[9-(4-chloro-phenyl)-8-(2-chloro-phenyl)-9H-purin-6-yl]-4-ethylamino-piperidine-4-carboxylic acid amide which can be prepared as described in U.S. Patent Publication No. 2004/0092520; 1-[7-(2-chloro-phenyl)-8-(4-chloro-phenyl)-2-methyl-pyrazolo[1,5-a][1,3,5]triazin-4-yl]-3-ethylamino-azetidine-3-carboxylic acid amide and 1-[7-(2-chloro-phenyl)-8-(4-chloro-phenyl)-2-methyl-pyrazolo[1,5-a][1,3,5]triazin-4-yl]-3-methylamino-azetidine-3-carboxylic acid amide which can be prepared as described in U.S. Patent Publication No. 2004/0157839; 3-(4-chloro-phenyl)-2-(2-chloro-phenyl)-6-(2,2-difluoro-propyl)-2,4,5,6-tetrahydro-pyrazolo[3,4-c]pyridin-7-one which can be prepared as described in U.S. Patent Publication No. 2004/0214855; 3-(4-chloro-phenyl)-2-(2-chloro-phenyl)-7-(2,2-difluoro-propyl)-6,7-dihydro-2H,5H-4-oxa-1,2,7-triaza-azulen-8-one which can be prepared as described in U.S. Patent Publication No. 2005/0101592; 2-(2-chloro-phenyl)-6-(2,2,2-trifluoro-ethyl)-3-(4-trifluoromethyl-phenyl)-2,6-dihydro-pyrazolo[4,3-d]pyrimidin-7-one which can be prepared as described in U.S. Patent Publication No. 2004/0214838; (S)-4-chloro-N-{[3-(4-chloro-phenyl)-4-phenyl-4,5-dihydro-pyrazol-1-yl]-methylamino-methylene}-benzenesulfonamide (SLV-319) and (S)-N-{[3-(4-chloro-phenyl)-4-phenyl-4,5-dihydro-pyrazol-1-yl]-methylamino-methylene}-4-trifluoromethyl-benzenesulfonamide (SLV-326) which can be prepared as described in PCT Patent Publication No. WO 02/076949; N-piperidino-5-(4-bromophenyl)-1-(2,4-dichlorophenyl)-4-ethylpyrazole-3-carboxamide which can be prepared as described in U.S. Pat. No. 6,432,984; 1-[bis-(4-chloro-phenyl)-methyl]-3-[(3,5-difluoro-phenyl)-methanesulfonyl-methylene]-azetidine which can be prepared as described in U.S. Pat. No. 6,518,264; 2-(5-(trifluoromethyl)pyridin-2-yloxy)-N-(4-(4-chlorophenyl)-3-(3-cyanophenyl)butan-2-yl)-2-methylpropanamide which can be prepared as described in PCT Patent Publication No. WO 04/048317; 4-{[6-methoxy-2-(4-methoxyphenyl)-1-benzofuran-3-yl]carbonyl}benzonitrile (LY-320135) which can be prepared as described in U.S. Pat. No. 5,747,524; 1-[2-(2,4-dichlorophenyl)-2-(4-fluorophenyl)-benzo[1,3]dioxole-5-sulfonyl]-piperidine which can be prepared as described in WO 04/013120; and β-amino-5-(4-chlorophenyl)-6-(2,4-dichlorophenyl)-furo[2,3-b]pyridin-2-yl]-phenyl-methanone which can be prepared as described in WO 04/012671.

Intestinal Inhibitors of the Microsomal Triglyceride Transfer Protein:

Microsomal Triglyceride Transfer Protein (MTP) catalyses the transporting of lipids between phospholipid surfaces. See, Wetterau J R et al., Biochim Biophys Acta 1345, 136-150 (1997). The protein is found in the lumen of liver and intestinal microsomes. MTP is a heterodimer which consists of an MTP-specific large subunit (97 kD) and protein disulphide isomerase (PDI, 58 kD). PDI is a widely distributed protein of the endoplasmatic reticulum (ER) and an essential component for the structural and functional integrity of MTP. MTP is necessary for the intracellular production of apolipoprotein B (apoB)-containing plasma lipoproteins. Although the precise role of MTP in the composition of the lipoproteins is not known, it most likely transports lipids from the membrane of the ER to the lipoprotein particles forming in the lumen of the ER. Apolipoprotein B is the main protein component of hepatic VLDL (very low density lipoproteins) and intestinal chylomicrons. Substances that inhibit MTP reduce the secretion of apoB-containing lipoproteins. Therefore, any inhibition of MTP lowers the plasma concentrations of cholesterol and triglycerides in apoB-containing lipoproteins. The inhibition of the intestinal absorption of fats from the food by MTP inhibitors is believed to be useful for treating conditions such as obesity and diabetes mellitus in which an excessive fat intake contributes significantly to the development of the disease. See, Grundy S M, Am J Clin Nutr 57(suppl), 563S-572S (1998).

In the practice of the present invention, the intestinal-acting MTP inhibitors are preferably intestinal selective. The term “intestinal selective” means that the MTP inhibitor has a higher exposure to the MTP in the intestinal microsomes than the MTP in the liver. Preferably, the MTPi is 3 fold more selective to the MTP in the intestinal microsomes than the MTP in the liver, more preferably, the MTPi is 10 fold more selective to the MTP in the intestinal microsomes than the MTP in the liver, most preferably, the MTPi is 100 fold more selective to the MTP in the intestinal microsomes than the MTP in the liver. Selectivity is generally measured by triglyceride (TG) accumulation. For example, useful intestinal-acting MTPi and/or doses of intestinal-acting MTPi are those that would lead to triglyceride accumulation in the intestine and do not result in statistically significant triglyceride accumulation in the liver. Triglyceride content would be assessed in animals by dissecting intestinal and hepatic tissue and extracting and quantitating triglyceride levels. Preferably, the TG accumulation in the intestine is 3 times more than the TG accumulation in the liver, more preferably, the TG accumulation in the intestine is 10 times more than TG accumulation in the liver, most preferably, the TG accumulation in the intestine is 100 times more than the TG accumulation in the liver. Since a correlation between TG accumulation in the intestine and reduction in food consumption was observed, it is reasonable to assume that reduction in food intake results either directly or indirectly from intestinal MTP inhibition; therefore, food intake measurements provide another useful means for evaluating intestinal MTP inhibition.

Intestinal selectivity may be achieved by controlling the solubility of the inhibitor in the intestinal tract and/or release of the inhibitor from the dosage form.

More recently, MTP inhibitors have been shown to reduce food intake in dogs and cats. See, EP1099438.

Suitable intestinal-acting MTP inhibitors include compounds disclosed in U.S. Pat. Nos. 4,453,913; 4,473,425; 4,491,589; 4,540,458; 4,962,115; 5,057,525; 5,137,896; 5,286,647; 5,521,186; 5,595,872; 5,646,162; 5,684,014; 5,693,650; 5,712,279; 5,714,494; 5,721,279; 5,739,135; 5,747,505; 5,750,783; 5,760,246; 5,789,197; 5,811,429; 5,827,875; 5,837,733; 5,849,751; 5,883,099; 5,883,109; 5,885,983; 5,892,114; 5,919,795; 5,922,718; 5,925,646; 5,929,075; 5,929,091; 5,935,984; 5,952,498; 5,962,440; 5,965,577; 5,968,950; 5,998,623; 6,025,378; 6,034,098; 6,034,115; 6,051,229; 6,051,387; 6,051,693; 6,057,339; 6,066,650; 6,066,653; 6,114,341; 6,121,283; 6,191,157; 6,194,424; 6,197,798; 6,197,972; 6,200,971; 6,235,730; 6,235,770; 6,245,775; 6,255,330; 6,265,431; 6,281,228; 6,288,234; 6,329,360; 6,342,245; 6,369,075; 6,417,362; 6,451,802; 6,479,503; 6,492,365; 6,583,144; 6,617,325; 6,713,489; 6,720,351; 6,774,236; and 6,777,414:

U.S. Patent Publication Nos. 2002/028940; 2002/032238; 2002/055635; 2002/132806; 2002/147209; 2003/149073; 2003/073836; 2003/105093; 2003/114442; 2003/0162788; 2003/166590; 2003/166637; 2003/181714; 2004/009988; 2004/014971; 2004/024215; 2004/034028; 2004/044008; 2004/058903; 2004/102490; 2004/157866; and 2005/234099:

PCT Patent Publication Nos. WO 96/262205; WO 98/016526; WO 98/031366; WO99/55313; WO 00/005201; WO 01/000183; WO 01/000184; WO 01/000189; WO 01/005767; WO 01/012601; WO 01/014355; WO 01/021604; WO 01/053260; WO 01/074817; WO 01/077077; WO 02/014276; WO 02/014277; WO 02/081460; WO 02/083658; and WO 04/017969: and

Japanese Patent Publication Nos. JP2002-212179(14212179); and JP2002-220345(14220345).

For a review of apo-B/MTP inhibitors, see, Williams, S. J. and J. D. Best, Expert Opin Ther Patents, 13(4), 479-488 (2003). For methods that may be used to identify active MTP inhibitors, see, Chang, G., et al., “Microsomal triglyceride transfer protein (MTP) inhibitors: Discovery of clinically active inhibitors using high-throughput screening and parallel synthesis paradigms,” Current Opinion in Drug Discovery & Development, 5(4), 562-570 (2002). All of the above patents, patent applications and references are incorporated herein by reference.

Preferred intestinal-acting MTP inhibitors for use in the combinations, pharmaceutical compositions, and methods of the invention include dirlotapide ((S)-N-{2-[benzyl(methyl)amino]-2-oxo-1-phenylethyl}-1-methyl-5-[4′-(trifluoromethyl)[1,1′-biphenyl]-2-carboxamido]-1H-indole-2-carboxamide) and 1-methyl-5-[(4′-trifluoromethyl-biphenyl-2-carbonyl)-amino]-1H-indole-2-carboxylic acid (carbamoyl-phenyl-methyl)-amide which can both be prepared using methods described in U.S. Pat. No. 6,720,351; (S)-2-[(4′trifluoromethyl-biphenyl-2-carbonyl)-amino]-quinoline-6-carboxylic acid (pentylcarbamoyl-phenyl-methyl)-amide, (S)-2-[(4′-tert-butyl-biphenyl-2-carbonyl)-amino]-quinoline-6-carboxylic acid {[(4-fluoro-benzyl)-methyl-carbamoyl]-phenyl-methyl}-amide, and (S)-2-[(4′-tert-butyl-biphenyl-2-carbonyl)-amino]-quinoline-6-carboxylic acid [(4-fluoro-benzylcarbamoyl)-phenyl-methyl]-amide which can all be prepared as described in U.S. Publication No. 2005/0234099; (−)-4-[4-[4-[4-[[(2S,4R)-2-(4-chlorophenyl)-2-[[(4-methyl-4H-1,2,4-triazol-3-yl)sulfanyl]methyl-1,3-dioxolan-4-yl]methoxy]phenyl]piperazin-1-yl]phenyl]-2-(1R)-1-methylpropyl]-2,4-dihydro-3H-1,2,4-triazol-3-one (also known as Mitratapide or R103757) which can be prepared as described in U.S. Pat. Nos. 5,521,186 and 5,929,075; and implitapide (BAY 13-9952) which can be prepared as described in U.S. Pat. No. 6,265,431. Most preferred is dirlotapide, mitratapide, (S)-2-[(4′-trifluoromethyl-biphenyl-2-carbonyl)-amino]-quinoline-6-carboxylic acid (pentylcarbamoyl-phenyl-methyl)-amide, (S)-2-[(4′-tert-butyl-biphenyl-2-carbonyl)-amino]-quinoline-6-carboxylic acid {[(4-fluoro-benzyl)-methyl-carbamoyl]-phenyl-methyl}-amide, or (S)-2-[(4′-tert-butyl-biphenyl-2-carbonyl)-amino]-quinoline-6-carboxylic acid [(4-fluoro-benzylcarbamoyl)-phenyl-methyl]-amide.

A typical formulation is prepared by mixing the CB-1 receptor antagonist and/or the intestinal-acting MTPi with a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include excipients such as buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug or aid in the manufacturing of the pharmaceutical product (i.e., medicament).

The formulations may be prepared using conventional dissolution and mixing procedures. For example, the bulk drug substance (the compound or stabilized form of the compound (e.g., complex with a cyclodextrin derivative or other known complexation agent)) is dissolved in a suitable solvent in the presence of one or more of the excipients described above. The compound is typically formulated into pharmaceutical dosage forms to provide an easily controllable dosage of the drug and to give the patient an elegant and easily handleable product. The CB-1 receptor antagonist and intestinal-acting MTPi may be formulated into a single dosage form or separate dosage forms. To enhance dissolution rates, it may be advantageous to disperse poorly water-soluble compounds in a suitable dispersant prior to formulating into a dosage form. For example, the water-insoluble or partially water-insoluble compound may be spray-dried in the presence of a solubilizing or dispersing agent. See, e.g., Takeuchi, Hirofumi, et al., J Pharm Pharmacol, 39, 769-773 (1987) and WO 05/046644. Other techniques for improving bioavailability of poorly water-soluble compounds are described in Verreck, G., et al., “The Use of Three Different solid Dispersion Formulations-Melt Extrusion, Film-coated Beads, and a Glass Thermoplastic System-to Improve the Bioavailability of a Novel Microsomal Triglyceride transfer Protein Inhibitor,” J Pharm Sci, 93(5), 1217-1228 (2004); and Peeters, J., et al., Proceed. Int'l. Symp. Control. Rel. Bioact. Mater., 28, 704-705 (2001).

For oral administration the pharmaceutical composition is generally administered in discrete units. For example, typical dosage forms include tablets, dragees, capsules, granules, sachets and liquid solutions or suspensions where each contain a predetermined amount of the active ingredient(s) in the form of a powder or granules, or as a solution or a suspension in an aqueous liquid, a non-aqueous liquid, an oil-in-water emulsion or a water-in-oil liquid emulsion.

Compressed tablets may be prepared by compressing the active ingredient(s) in a free-flowing form such as a powder or granules with a binder, lubricant, inert diluent, surface active agent and/or dispersing agent.

Liquid dosage forms for oral administration include pharmaceutically acceptable emulsions, solutions, suspensions, syrups, and elixirs. In addition to the active ingredient(s), the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed oil, groundnut oil, corn germ oil, olive oil, castor oil, sesame seed oil and the like), glycerol, tetrahydrofurfuryl alcohol, polyethylene glycols and fatty acid esters of sorbitan, or mixtures of these substances, and the like.

Besides such inert diluents, the composition can also include excipients, such as wetting agents, emulsifying and suspending agents, sweetening, and flavoring agents.

Suspensions, in addition to the active ingredients, may further comprise suspending agents, e.g., ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings. The container may also contain instructions on using the dosage form(s) for treatment of obesity or related eating disorders, or for reduction of food consumption.

The compounds can be administered by any method which delivers the compounds preferentially to the desired tissue (e.g., brain, renal or intestinal tissues). These methods include oral routes, parenteral, intraduodenal routes, transdermal, etc. Generally, the compounds are administered orally in single (e.g., once daily) or multiple doses. The amount and timing of compounds administered will, of course, be dependent on the subject being treated, on the severity of the affliction, on the manner of administration and on the judgment of the prescribing physician. Thus, because of patient to patient variability, the dosages given herein are a guideline and the physician may titrate doses of the drug to achieve the treatment that the physician considers appropriate for the patient. In considering the degree of treatment desired, the physician must balance a variety of factors such as age of the patient, presence of preexisting disease, lifestyle, as well as presence of other diseases (e.g., cardiovascular disease).

For human use, the daily dose of the intestinal-acting MTPi is generally between about 0.05 mg to about 50 mg, preferably between about 0.5 mg to about 30 mg, more preferably between about 0.5 mg to about 20 mg, most preferably between about 1.0 mg to about 15 mg. For non-human use, those skilled in the art know how to adjust the dosage for the particular weight of the animal. In some circumstances, the MTPi may be administered in combination with an agent to reduce fatty liver (e.g., fibrate or PPAR-alpha agonist). See, e.g., JP Publication No. 2002-220345 (Application No. 2001-015602) entitled “Remedial Agent for Fatty Liver”; and Kersten, S., “Peroxisome Proliferator Activated Receptors and Obesity,” Eur J Pharm, 440, 223-234 (2002).

For human use, the daily dose of the CB-1 receptor antagonist is generally between about 1.0 mg to about 100 mg, preferably between about 1.0 mg to about 50 mg, more preferably between about 2.0 mg to about 40 mg, most preferably between about 5.0 mg to about 25 mg. For non-human use, those skilled in the art know how to adjust the dosage for the particular weight of the animal.

### Pharmacological Testing

### Identification of CB-1 Antagonists

CB-1 antagonists that are useful in the practice of the instant invention can be identified using at least one of the protocols described hereinbelow. The following acronyms are used in the protocols described below.

BSA—bovine serum albumin

DMSO—dimethylsulfoxide

EDTA—ethylenediamine tetracetic acid

PBS—phosphate-buffered saline

EGTA—ethylene glycol-bis(β-aminoethyl ether) N,N,N′,N′-tetraacetic acid

GDP—guanosine diphosphate

[³H]SR141716A—radiolabeled N-(piperidin-1-yl)-5-(4-chlorophenyl)-1-(2,4-dichlorophenyl)-4-methyl-1H-pyrazole-3-carboxamide hydrochloride available from Amersham Biosciences, Piscataway, N.J.

[³H]CP-55940—radiolabled 5-(1,1-dimethylheptyl)-2-[5-hydroxy-2-(3-hydroxypropyl)-cyclohexyl]-phenol available from NEN Life Science Products, Boston, Mass.

AM251—N-(piperidin-1-yl)-1-(2,4-dichlorophenyl)-5-(4-iodophenyl)-4-methyl-1H-pyrazole-3-carboxamide available from Tocris™, Ellisville, Mo.

### In Vitro Biological Assays

Bioassay systems for determining the CB-1 and CB-2 binding properties and pharmacological activity of cannabinoid receptor ligands are described by Roger G. Pertwee in “Pharmacology of Cannabinoid Receptor Ligands” Current Medicinal Chemistry, 6, 635-664 (1999) and in WO 92/02640 (U.S. application Ser. No. 07/564,075 filed Aug. 8, 1990, incorporated herein by reference).

The following assays are designed to detect compounds that inhibit the binding of [³H] SR141716A (selective radiolabeled CB-1 ligand) and [³H] 5-(1,1-dimethylheptyl)-2-[5-hydroxy-2-(3-hydroxypropyl)-cyclohexyl]-phenol ([³H] CP-55940; radiolabeled CB-1/CB-2 ligand) to their respective receptors.

### Rat CB-1 Receptor Binding Protocol

PelFreeze brains (available from Pel Freeze Biologicals, Rogers, Ark.) are cut up and placed in tissue preparation buffer (5 mM Tris HCl, pH=7.4 and 2 mM EDTA), polytroned at high speed and kept on ice for 15 minutes. The homogenate is then spun at 1,000×g for 5 minutes at 4° C. The supernatant is recovered and centrifuged at 100,000×G for 1 hour at 4° C. The pellet is then re-suspended in 25 ml of TME (25 nM Tris, pH=7.4, 5 mM MgCl₂, and 1 mM EDTA) per brain used. A protein assay is performed and 200 μl of tissue totaling 20 μg is added to the assay.

The test compounds are diluted in drug buffer (0.5% BSA, 10% DMSO and TME) and then 25 μl are added to a deep well polypropylene plate. [³H] SR141716A is diluted in a ligand buffer (0.5% BSA plus TME) and 25 μl are added to the plate. A BCA protein assay is used to determine the appropriate tissue concentration and then 200 μl of rat brain tissue at the appropriate concentration is added to the plate. The plates are covered and placed in an incubator at 20° C. for 60 minutes. At the end of the incubation period, 250 μl of stop buffer (5% BSA plus TME) is added to the reaction plate. The plates are then harvested by Skatron onto GF/B filtermats presoaked in BSA (5 mg/ml) plus TME. Each filter is washed twice. The filters are dried overnight. In the morning, the filters are counted on a Wallac Betaplate™ counter (available from PerkinElmer Life Sciences™, Boston, Mass.).

### Human CB-1 Receptor Binding Protocol

Human embryonic kidney 293 (HEK 293) cells transfected with the CB-1 receptor cDNA (obtained from Dr. Debra Kendall, University of Connecticut) are harvested in homogenization buffer (10 mM EDTA, 10 mM EGTA, 10 mM Na Bicarbonate, protease inhibitors; pH=7.4), and homogenized with a Dounce Homogenizer. The homogenate is then spun at 1,000×g for 5 minutes at 4° C. The supernatant is recovered and centrifuged at 25,000×G for 20 minutes at 4° C. The pellet is then re-suspended in 10 ml of homogenization buffer and re-spun at 25,000×G for 20 minutes at 4° C. The final pellet is re-suspended in 1 ml of TME (25 mM Tris buffer (pH=7.4) containing 5 mM MgCl₂ and 1 mM EDTA). A protein assay is performed and 200 μl of tissue totaling 20 μg is added to the assay.

The test compounds are diluted in drug buffer (0.5% BSA, 10% DMSO and TME) and then 25 μl are added to a deep well polypropylene plate. [3H] SR141716A is diluted in a ligand buffer (0.5% BSA plus TME) and 25 μl are added to the plate. The plates are covered and placed in an incubator at 30° C. for 60 minutes. At the end of the incubation period, 250 μl of stop buffer (5% BSA plus TME) is added to the reaction plate. The plates are then harvested by Skatron onto GF/B filtermats presoaked in BSA (5 mg/ml) plus TME. Each filter is washed twice. The filters are dried overnight. In the morning, the filters are counted on a Wallac Betaplate™ counter (available from PerkinElmer Life Sciences™, Boston, Mass.).

### CB-2 Receptor Binding Protocol

Chinese hamster ovary-K1 (CHO-K1) cells transfected with CB-2 cDNA (obtained from Dr. Debra Kendall, University of Connecticut) are harvested in tissue preparation buffer (5 mM Tris-HCl buffer (pH=7.4) containing 2 mM EDTA), polytroned at high speed and kept on ice for 15 minutes. The homogenate is then spun at 1,000×g for 5 minutes at 4° C. The supernatant is recovered and centrifuged at 100,000×G for 1 hour at 4° C. The pellet is then re-suspended in 25 ml of TME (25 mM Tris buffer (pH=7.4) containing 5 mM MgCl₂ and 1 mM EDTA) per brain used. A protein assay is performed and 200 μl of tissue totaling 10 μg is added to the assay.

The test compounds are diluted in drug buffer (0.5% BSA, 10% DMSO, and 80.5% TME) and then 25 μl are added to the deep well polypropylene plate. [3H] CP-55940 is diluted a ligand buffer (0.5% BSA and 99.5% TME) and then 25 μl are added to each well at a concentration of 1 nM. A BCA protein assay is used to determine the appropriate tissue concentration and 200 μl of the tissue at the appropriate concentration was added to the plate. The plates are covered and placed in an incubator at 30° C. for 60 minutes. At the end of the incubation period 250 μl of stop buffer (5% BSA plus TME) is added to the reaction plate. The plates are then harvested by Skatron format onto GF/B filtermats presoaked in BSA (5 mg/ml) plus TME. Each filter is washed twice. The filters are dried overnight. The filters are then counted on the Wallac Betaplate™ counter.

### CB-1 GTPγ[³⁵S] Binding Assay

Membranes are prepared from CHO-K1 cells stably transfected with the human CB-1 receptor cDNA. Membranes are prepared from cells as described by Bass et al, in “Identification and characterization of novel somatostatin antagonists,” Molecular Pharmacology, 50, 709-715 (1996). GTPγ [³⁵S] binding assays are performed in a 96 well FlashPlate™ format in duplicate using 100 pM GTPγ[³⁵S] and 10 μg membrane per well in assay buffer composed of 50 mM Tris HCl, pH 7.4, 3 mM MgCl₂, pH 7.4, 10 mM MgCl₂, 20 mM EGTA, 100 mM NaCl, 30 μM GDP, 0.1% bovine serum albumin and the following protease inhibitors: 100 μg/ml bacitracin, 100 μg/ml benzamidine, 5 μg/ml aprotinin, 5 μg/ml leupeptin. The assay mix is then incubated with increasing concentrations of antagonist (10⁻¹⁰ M to 10⁻⁵ M) for 10 minutes and challenged with the cannabinoid agonist CP-55940 (10 μM). Assays are performed at 30° C. for one hour. The FlashPlates™ are then centrifuged at 2000×g for 10 minutes. Stimulation of GTPγ[³⁵S] binding is then quantified using a Wallac Microbeta.EC₅₀ calculations done using Prism™ by Graphpad.

Inverse agonism is measured in the absense of agonist.

### CB-1 FLIPR-Based Functional Assay Protocol

CHO-K1 cells co-transfected with the human CB-1 receptor cDNA (obtained from Dr. Debra Kendall, University of Connecticut) and the promiscuous G-protein G16 are used for this assay. Cell's are plated 48 hours in advance at 12500 cells per well on collagen coated 384 well black clear assay plates. Cells are incubated for one hour with 4 μM Fluo-4 AM (Molecular Probes) in DMEM (Gibco) containing 2.5 mM probenicid and pluronic acid (0.04%). The plates are then washed 3 times with HEPES-buffered saline (containing probenicid; 2.5 mM) to remove excess dye. After 20 minutes, the plates are added to the FLIPR individually and fluorescence levels are continuously monitored over an 80 second period. Compound additions are made simultaneously to all 384 wells after 20 seconds of baseline. Assays are performed in triplicate and 6 point concentration-response curves generated. Antagonist compounds are subsequently challenged with 3 μM WIN 55,212-2 (agonist). Data is analyzed using Graph Pad Prism.

### Detection of Inverse Agonists

The following cyclic-AMP assay protocol using intact cells may be used to determine inverse agonist activity.

Cells are plated into a 96-well plate at a plating density of 10,000-14,000 cells per well at a concentration of 100 μl per well. The plates are incubated for 24 hours in a 37° C. incubator. The media is removed and media lacking serum (100 μl) is added. The plates are then incubated for 18 hours at 37° C.

Serum free medium containing 1 mM IBMX is added to each well followed by 10 μl of test compound (1:10 stock solution (25 mM compound in DMSO) into 50% DMSO/PBS) diluted 10× in PBS with 0.1% BSA. After incubating for 20 minutes at 37° C., 2 μM of Forskolin is added and then incubated for an additional 20 minutes at 37° C. The media is removed, 100 μl of 0.01 N HCl is added and then incubated for 20 minutes at room temperature. Cell lysate (75 μl) along with 25 μl of assay buffer (supplied in FlashPlate™ cAMP assay kit available from NEN Life Science Products Boston, Mass.) into a Flashplate. cAMP standards and cAMP tracer is added following the kit's protocol. The flashplate is then incubated for 18 hours at 4° C. The content of the wells are aspirated and counted in a Scintillation counter.

### Identification of Intestinal-Acting MTPi

Intestinal-acting MTPi that are useful in the practice of the instant invention can be identified using the protocol described hereinbelow. The following reagents used in the protocols described below may be purchased from the corresponding suppliers.

Triton-X™ 100 is a non-ionic surfactant available from Union Carbide Chemicals & Plastics Technology Corp.

Aprotinin is available from Apollo Scientific Ltd, United Kingdom.

WAKO Triglyceride L-Type Colorimetric assay is available from Waco Chemicals, Richmond, Va.

### Apo B Secretion Inhibition

The ability of the compounds of the present invention to inhibit the secretion of apo B was determined using the following cell-based assay, which measures the secretion of apo B in HepG2 cells.

HepG2 cells (ATCC, HB-8065, Manassas, Va.) were grown in Dulbecco's Modified Eagles Medium plus 10% fetal bovine serum (Growth medium; Gibco, Grand Island, N.Y.) in 96-well culture plates in a humidified atmosphere containing 5% carbon dioxide until they were approximately 70% confluent. Test compounds were dissolved at 10 mM in dimethyl sulfoxide (DMSO). From this stock, the initial dose concentration was prepared in 70% EtOH and subsequent serial dilutions made in 70% EtOH with DMSO at a concentration equivalent to the initial dilution. Dilutions of test compounds were prepared at 100× the desired final concentation and were added in triplicate to separate wells of a 96-well culture plate containing HepG2 cells. Forty hours later, growth medium was collected and assayed by specific enzyme-linked immunosorbent assay (ELISA) for Apo B. Inhibitors were identified as compounds that decrease Apo B secretion into the medium. The ELISA assay for Apo B was performed as follows: Polyclonal antibody against human Apo B (Chemicon, Temecula, Calif.) is diluted 1:1000 in carbonate-bicarbonate buffer (Pierce, Rockford, Ill.) and 100 μL was added to each well of a 96-well plate (NUNC Maxisorb, Rochester, N.Y.). After 5 hours incubation at room temperature, the antibody solution was removed and wells were washed four times with phosphate buffered saline (PBS)/0.05% Tween® 20 (Tween® 20 is available from Cayman Chemical Co., Ann Arbor Mich.). Non-specific sites on the plastic were blocked by incubating wells for 1 to 1.5 hours in a solution of 0.5% (w/v) bovine serum albumin (BSA), 0.1% Tween® 20 made in PBS. One hundred microliters (100 μL) of a 1:20 dilution of growth medium from the HepG2 cells (made in 0.004% Tween® 20/1% BSA in PBS) were added to each well and incubated for 3 hours at room temperature. Wells were aspirated and washed four times (0.05% Tween® 20 in PBS) prior to adding 100 μL of a 1/1000 dilution (˜5 ug/mL) of the secondary antibody, mouse anti-human Apo B (Chemicon, Temecula, Calif.). After 2 hours incubation at room temperature, this solution was aspirated and the wells were again washed 4 times as above. One hundred microliters (100 μL) of a 1:10,000 dilution (0.004% Tween® 20/1% BSA in PBS) of peroxidase-conjugated affinpure goat anti-mouse IgG (H+L) (Jackson ImmunoResearch Laboratories, Bar Harbor, Me.)) were then added to each well and incubated for 1 hour at room temperature. After aspirating, the wells were washed 4 times as above and 50 μl of 1-step Ultra TMB (tetramethylbenzidine) ELISA reagent (Pierce, Rockford, Ill.) was added to each well and incubated for 5 minutes. The reaction was stopped by the addition of 50 μL of 2M H₂SO₄ and absorbance of each well was read at 450 nm. Percent inhibition was calculated using absorbance from vehicle-treated supernatants minus the absorbance from media alone as the total or 100% value. The percent inhibition at each concentration of test compound was recorded and IC₅₀ values were determined.

### Food Intake, Body Weight and Triglyceride Accumulation

The effect of an MTP inhibitor on food intake in male Sprague Dawley rats (available from Charles River Laboratories) was evaluated by feeding the rats either a low or high fat diet following 3 daily oral doses of 0, 10, 30 and 100 mg/kg of test compound in a 0.5% methylcellulose vehicle. The endpoints measured include food intake, body weight, and liver and/or intestinal triglycerides.

Powdered high fat experimental diet with 45% fat and cornstarch/maltodextrin for carbohydrate (Research Diets D01060502M) was used. Rats were weighed on days 0 and 3. Food intake was measured daily on day −4 to 3. At the time of euthanasia on day 3, blood was collected and placed into EDTA tubes (75%) containing Aprotinin (0.6 TIU/mL) and serum separator tubes (25%) and stored frozen, an approximately 0.5 g piece of liver tissue was removed, rinsed with sterile saline, weighed and frozen in liquid nitrogen.

For determination of liver triglyceride, liver pieces were homogenized in PBS, and an aliquot was extracted with chloroform:methanol (2:1). The dried extracts were reconstituted with Triton-X™ 100 in absolute ethanol and an aliquot was used for triglyceride analysis using a WAKO Triglyceride L-Type Colorimetric assay (Cat # 997-37492 Enzyme A, Cat # 993-37592, Cat # 99641791 Lipids Calibrator). An analogous method well-known to those of skill in the art was used for assessing intestinal triglyceride content.

### EXAMPLES

The following compounds and reagents used in the experiments illustrated below may be prepared as described in the listed disclosures or available from the listed vendors.

Dirlotapide: ((S)-N-{2-[benzyl(methyl)amino]-2-oxo-1-phenylethyl}-1-methyl-5-[4′-(trifluoromethyl)[1,1′-biphenyl]-2-carboxamido]-1H-indole-2 carboxamide) was prepared using methods described in U.S. Pat. No. 6,720,351 (Example 44).

Compound A: 1-[9-(4-chloro-phenyl)-8-(2-chloro-phenyl)-9H-purin-6-yl]4-ethylamino-piperidine-4-carboxylic acid amide Hydrochloride salt was prepared as described in U.S. Patent Publication No. 2004/0092520 (Example 20).

Miglyol® 812: a fractionated coconut oil having a boiling range of 240-270° C. and composed of saturated C₈ (50-65%) and C₁₀ (30-45%) triglycerides, available from CONDEA Vista Co., Cranford, N.J.

Triacetin®: Glyceryl triacetate available from Sigma-Aldrich, St. Louis, Mo.

Tween® 80: Polysorbate 80 available from Sigma-Aldrich, St. Louis, Mo.

Capmul® MCM: Medium chain mono- & diglycerides, available from ABITEC Corporation, Columbus, Ohio.

The following functional assay was used to determine the effect of an intestinal-acting MTPi, a CB-1 antagonist, and the combination of an intestinal-acting MTPi and a CB-1 antagonist on food intake. The doses of the CB-1 antagonist used in the experiments were 10 mg/kg and 30 mg/kg. The doses of the intestinal-acting MTPi used in the experiments were 3 mg/kg and 10 mg/kg. The different dosages for each active were tested alone and in various combinations with each other as compared to a control (vehicle).

### Food Intake

Male Sprague-Dawley rats (275-325 grams) were placed on a high fat diet (Research Diets, 45% kcal from fat). Animals were acclimated to an automated food intake assessment system overnight. Food weight data was collected by computer acquisition. Immediately prior to the start of the dark cycle on the first day, animals were given a PO (i.e., orally by mouth) dose of a gMTP inhibitor (Dirlotapide) or vehicle (self-emulsifying drug delivery system (SEDDS) formulation containing 20% Miglyol 812, 30% Triacetin, 20% Tween 80, and 30% Capmul MCM). On the second day, rats (n=5-10/group) were given a PO dose of a CB-1 antagonist (Compound A) or 0.5% methylcellulose 20 minutes prior to a second dose of Dirlotapide or vehicle. Food Intake was monitored until the following day. Data for each treatment group was compared by ANOVA (analysis of variance).

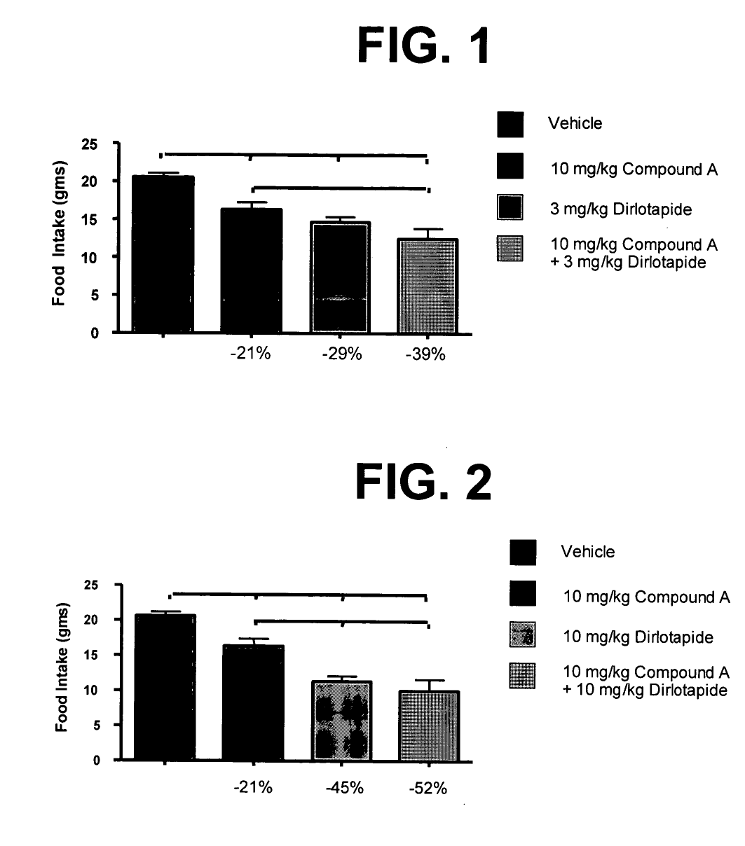

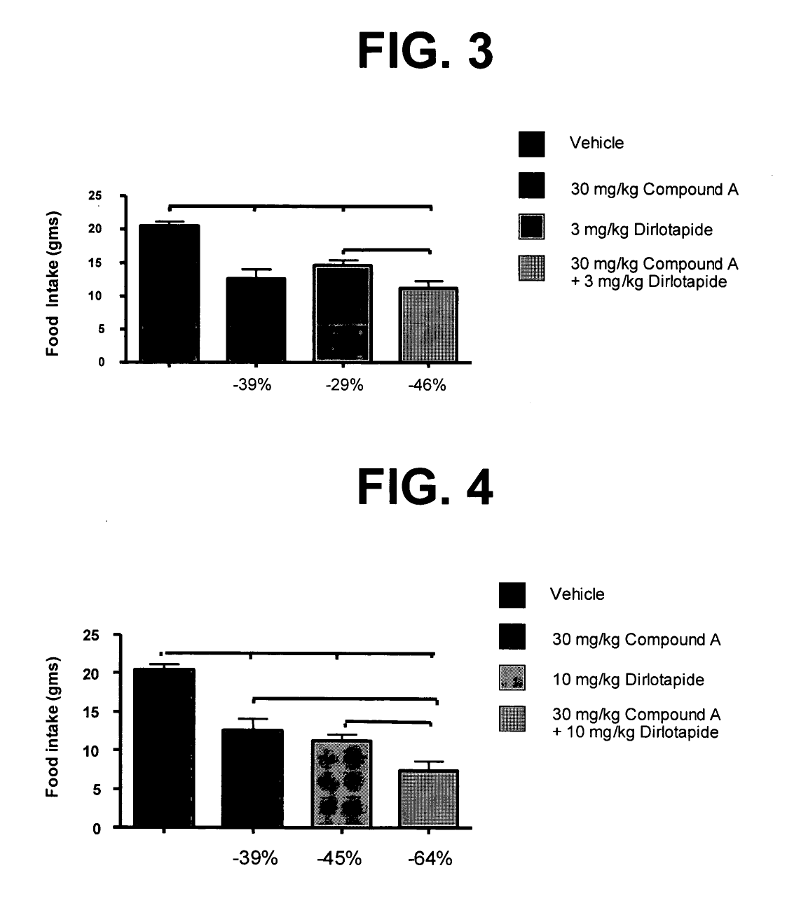

The results observed for food intake are summarized below in Table 1 and graphically depicted in FIGS. 1, 2, 3 and 4.

TABLE 1
12 hour Spontaneous Food Intake (grams)

| | | 10 mg/kg | 30 mg/kg |
|---|---|---|---|
| | VEH | Compound A | Compound A |
| Vehicle (VEH) | 20.5 ± 0.6 | 16.3 ± 1.0 | 12.6 ± 1.4 |
| 3 mg/kg | 14.6 ± 0.8 | 12.5 ± 1.4 | 11.2 ± 1.1 |
| Dirlotapide | | | |
| 10 mg/kg | 11.3 ± 0.7 | 9.9 ± 1.7 | 7.4 ± 1.2 |
| Dirlotapide | | | |

What is claimed is:

1. A method for treating obesity and related eating disorders comprising the step of administering a therapeutically effective amount of a combination comprising a cannabinoid-1 receptor antagonist and an intestinal-acting microsomal triglyceride transfer protein inhibitor to an animal in need of such treatment.

2. A method for reducing food consumption comprising comprising the step of administering a therapeutically effective amount of a combination comprising a cannabinoid-1 receptor antagonist and an intestinal-acting microsomal triglyceride transfer protein inhibitor to an animal in need of such treatment.

3. The method of claim 1 or 2 wherein said cannabinoid-1 receptor antagonist is selected from the group consisting of
rimonabant;
N-(piperidin-1-yl)-1-(2,4-dichlorophenyl)-5-(4-iodophenyl)-4-methyl-1H-pyrazole-3-carboxamide;
[5-(4-bromophenyl)-1-(2,4-dichloro-phenyl)-4-ethyl-N-(1-piperidinyl)-1H-pyrazole-3-carboxamide];
N-(piperidin-1-yl)-4,5-diphenyl-1-methylimidazole-2-carboxamide;
N-(piperidin-1-yl)-4-(2,4-dichlorophenyl)-5-(4-chlorophenyl)-1-methylimidazole-2-carboxamide;
N-(piperidin-1-yl)-4,5-di-(4-methylphenyl)-1-methylimidazole-2-carboxamide;
N-cyclohexyl-4,5-di-(4-methylphenyl)-1-methylimidazole-2-carboxamide;
N-(cyclohexyl)-4-(2,4-dichlorophenyl)-5-(4-chlorophenyl)-1-methylimidazole-2-carboxamide;
N-(phenyl)-4-(2,4-dichlorophenyl)-5-(4-chlorophenyl)-1-methylimidazole-2-carboxamide;
1-[9-(4-chloro-phenyl)-8-(2-chloro-phenyl)-9H-purin-6-yl]-4-ethylamino-piperidine-4-carboxylic acid amide, or a pharmaceutically acceptable salt thereof;
1-[7-(2-chloro-phenyl)-8-(4-chloro-phenyl)-2-methyl-pyrazolo[1,5-a][1,3,5]triazin-4-yl]-3-ethylamino-azetidine-3-carboxylic acid amide;
1-[7-(2-chloro-phenyl)-8-(4-chloro-phenyl)-2-methyl-pyrazolo[1,5-a][1,3,5]triazin-4-yl]-3-methylamino-azetidine-3-carboxylic acid amide;
3-(4-chloro-phenyl)-2-(2-chloro-phenyl)-6-(2,2-difluoro-propyl)-2,4,5,6-tetrahydro-pyrazolo[3,4-c]pyridin-7-one;
3-(4-chloro-phenyl)-2-(2-chloro-phenyl)-7-(2,2-difluoro-propyl)-6,7-dihydro-2H,5H4-oxa-1,2,7-triaza-azulen-8-one;
2-(2-chloro-phenyl)-6-(2,2,2-trifluoro-ethyl)-3-(4-trifluoromethyl-phenyl)-2,6-dihydro-pyrazolo[4,3-d]pyrimidin-7-one;
(S)₄-chloro-N-{[3-(4-chloro-phenyl)₄-phenyl-4,5-dihydro-pyrazol-1-yl]-methylamino-methylene}-benzenesulfonamide;
(S)-N-{[3-(4-chloro-phenyl)-4-phenyl-4,5-dihydro-pyrazol-1-yl]-methylamino-methylene}-4-trifluoromethyl-benzenesulfonamide;
N-piperidino-5-(4-bromophenyl)-1-(2,4-dichlorophenyl)-4-ethylpyrazole-3-carboxamide;
1-[bis-(4-chloro-phenyl)-methyl]-3-[(3,5-difluoro-phenyl)-methanesulfonyl-methylene]-azetidine;
2-(5-(trifluoromethyl)pyridin-2-yloxy)-N-(4-(4-chlorophenyl)-3-(3-cyanophenyl)butan-2-yl)-2-methylpropanamide;
4-{[6-methoxy-2-(4-methoxyphenyl)-1-benzofuran-3-yl]carbonyl}benzonitrile;
1-[2-(2,4-dichlorophenyl)-2-(4-fluorophenyl)-benzo[1,3]dioxole-5-sulfonyl]-piperidine; and
[3-amino-5-(4-chlorophenyl)-6-(2,4-dichlorophenyl)-furo[2,3-b]pyridin-2-yl]-phenyl-methanone;
or a pharmaceutically acceptable hydrate or solvate thereof.

4. The method of claim 1 or 2 wherein said intestinal-acting microsomal triglyceride transfer protein inhibitor is selected from the group consisting of
dirlotapide;
mitratapide;
1-methyl-5-[(4′-trifluoromethyl-biphenyl-2-carbonyl)-amino]-1H-indole-2-carboxylic acid (carbamoyl-phenyl-methyl)-amide;
(S)-2-[(4′-trifluoromethyl-biphenyl-2-carbonyl)-amino]-quinoline-6-carboxylic acid (pentylcarbamoyl-phenyl-methyl)-amide;
(S)-2-[(4′-tert-butyl-biphenyl-2-carbonyl)-amino]-quinoline-6-carboxylic acid {[(4-fluoro-benzyl)-methyl-carbamoyl]-phenyl-methyl}-amide;
(S)-2-[(4′-tert-butyl-biphenyl-2-carbonyl)-amino]-quinoline-6-carboxylic acid [(4-fluoro-benzylcarbamoyl)-phenyl-methyl]-amide;
4-(4-(4-(4-((2-((4-methyl-4H-1,2,4-triazol-3-ylthio)methyl)-2-(4-chlorophenyl)-1,3-dioxolan-4-yl)methoxy)phenyl)piperazin-1-yl)phenyl)-2-sec-butyl-2H-1,2,4-triazol-3(4H)-one; and
implitapide;
or a pharmaceutically acceptable hydrate or solvate thereof.

5. The method of claim 4 wherein said combination comprises from about 1.0 mg to about 100 mg of said cannabinoid-1 receptor antagonist.

6. The method of claim 4 wherein said combination comprises from about 0.05 mg to about 50 mg of intestinal-acting microsomal triglyceride transfer protein inhibitor.

7. The method of claim 1 or 2 wherein said cannabinoid-1 receptor antagonist and said intestinal-acting microsomal triglyceride transfer protein inhibitor are administered as a single pharmaceutical composition comprising said cannabinoid-1 receptor antagonist, said intestinal-acting microsomal triglyceride transfer protein inhibitor, and a pharmaceutically acceptable excipient, diluent, or carrier.

8. The method of claim 1 or 2 wherein said cannabinoid-1 receptor antagonist and said intestinal-acting microsomal triglyceride transfer protein inhibitor are administered as two separate pharmaceutical compositions comprising
(i) a first composition comprising said cannabinoid-1 receptor antagonist and a pharmaceutically acceptable excipient, diluent, or carrier, and
(ii) a second composition comprising said intestinal-acting microsomal triglyceride transfer protein inhibitor and a pharmaceutically acceptable excipient, diluent, or carrier.

9. A pharmaceutical composition (i) a CB-1 receptor antagonist; (ii) a intestinal-acting MTPi; and (iii) a pharmaceutically acceptable excipient, diluent, or carrier, wherein the amount of CB-1 receptor antagonist is from about 1.0 mg to about 100 mg and the amount of intestinal-acting MTPi is from about 0.05 mg to about 50 mg.
